(19) United States
(12) Patent Application Publication
(10) Pub. No.: US 2024/0293828 A1
(43) Pub. Date: Sep. 5, 2024

(54) CENTRIFUGAL STRUCTURE MEMBER OF MICROFLUIDIC CHIP AND CENTRIFUGE

(71) Applicants: BEIJING BOE TECHNOLOGY DEVELOPMENT CO., LTD., BEIJING (CN); BOE TECHNOLOGY GROUP CO., LTD., BEIJING (CN)

(72) Inventors: Kang PENG, BEIJING (CN); Zhukai LIU, BEIJING (CN); Ding DING, BEIJING (CN)

(73) Assignees: BEIJING BOE TECHNOLOGY DEVELOPMENT CO., LTD., BEIJING (CN); BOE TECHNOLOGY GROUP CO., LTD., BEIJING (CN)

(21) Appl. No.: 17/996,129

(22) PCT Filed: Oct. 22, 2021
(86) PCT No.: PCT/CN2021/125687

§ 371 Date: Oct. 13, 2022

(30) Foreign Application Priority Data
May 27, 2021 (CN) 202121157577.5

(51) Int. Cl. B04B 7/08; B01L 3/00

(52) U.S. Cl. CPC B04B 7/08; B01L 3/502707; B01L 2400/0409

(57) ABSTRACT

A centrifugal structure member of a microfluidic chip and a centrifuge are provided. The centrifugal structure member of a microfluidic chip includes: a connecting portion (10) configured to connect to a rotor of a centrifuge; a support portion (20) fixedly connected or integrally formed with the connecting portion (10), and having an inclined outer surface forming an included angle with the rotation axis (50) of the rotor of the centrifuge; and a mounting portion (30) connected with or formed on the inclined outer surface, and configured to detachably mount the microfluidic chip (40) on the support portion (20).

### CROSS-REFERENCE TO RELATED APPLICATIONS

The present application is a U. S. National Stage Application under 35 U.S. C. § 371 of International Patent Application No. PCT/CN2021/125687, filed on Oct. 22, 2021, which is based on and claims priority to China Patent Application No. 202121157577.5 filed on May 27, 2021, the disclosure of both which are incorporated by reference herein in its entirety.

### TECHNICAL FIELD

The present disclosure relates to the technical field of a microfluidic chip, and in particular, to a centrifugal structure member of a microfluidic chip and a centrifuge.

### BACKGROUND

The microfluidic chip technology is a process of realizing a complex biochemical reaction in a small-sized chip based on micron-scale fluid manipulation, which makes it possible for large-scale analytical instruments to gradually perform iterative upgrading towards directions such as miniaturization, integration, automation and high-flux. At present, there are many biochemical reactions that can be integrated in a microfluidic chip, one of which is Polymerase Chain Reaction (simply referred to as PCR). Such technology is a classic molecular biology experimental technology that synthesizes a large number of target DNA fragments by in-vitro enzymatic reaction, with the characteristics such as distinctive specificity, high sensitivity and easy operation, which is widely applied in fields such as gene cloning, sequence analysis, disease diagnosis and pathogen detection.

The digital PCR is the third-generation nucleic acid molecule quantitative analysis technology that has developed rapidly in recent years, with such principles that one sample is evenly distributed to several ten thousand different reaction units, each unit contains at least one copy of a target DNA template, and then PCR amplification is performed in each reaction unit respectively, and statistical analysis is performed on a fluorescence signal of each reaction unit after the amplification is concluded. The digital PCR is mainly implemented in the forms of arrays and droplets.

In the related art, the microarray digital PCR chip mainly includes a glass cover plate containing two sample injection holes and a substrate containing a microarray chamber, wherein the glass cover plate and the substrate are encapsulated into a complete chip by UV curing adhesive.

### SUMMARY

The inventors have found by studies that, when the microarray digital PCR chip in the related art performs sample injection, the PCR reaction solution and the oil phase enter the microarray chamber through one of the sample injection holes in the upper layer sequentially, and subsequently excessive liquid flows out another from hole. Due to uneven degrees of hydrophilicity and hydrophobicity at various locations of the surface of the chamber, it is extremely easily to form bubbles in the chamber area during the sample injection process, and once the liquid flows out from the hole at the other end, the bubbles are difficult to be discharged, which results in that the reaction liquid enters various microarray chambers in low efficiency so that it is often impossible to fill up the entire microarray chamber smoothly. Moreover, these bubbles directly affect the sample amplification efficiency and result interpretation, which limits the application of an array digital PCR chip. In addition, during the subsequent heating and cooling processes, with the nature of thermal expansion and contracting, the bubbles are easily to move around the surface of the array, which results in crosstalk of the reaction solutions between the chambers and ultimately affects the PCR amplification efficiency.

In view of this, the embodiments of the present disclosure provide a centrifugal structure member of a microfluidic chip and a centrifuge, which can reduce or eliminate the air bubbles within the chamber of the microfluidic chip.

In one aspect of the present disclosure, a centrifugal structure member of a: microfluidic chip is provided. The centrifugal structure member includes: a connecting portion configured to connect a rotor of a centrifuge; a support portion fixedly connected or integrally formed with the connecting portion, and having an inclined outer surface forming an included angle with a rotation axis of the rotor of the centrifuge; and a mounting portion connected with or formed on the inclined outer surface, and configured to detachably mount the microfluidic chip on the support portion.

In some embodiments, the mounting portion includes: a chip groove formed on the inclined outer surface, and configured to be embedded into by the microfluidic chip.

In some embodiments, the mounting portion further includes: a limiting protrusions connected with outside of the chip groove or formed beyond the chip groove and extending toward the chip groove, wherein a projection of the limiting protrusion on a flat surface of groove bottom of the chip groove is partially coincident with the groove bottom of the chip groove.

In some embodiments, the chip groove is rectangular and has two first side edges perpendicular to the rotation axis and opposite to each other, and two second side edges perpendicular to the two first side edges and opposite to each other, and the height of a first one of the two first side edges relative to the groove bottom of the chip groove is lower than that of a second one of the two first side edges relative to the groove bottom.

In some embodiments, the first one of the two first side edges is flush with a top end of the support portion.

In some embodiments, the mounting portion further includes: at least two limiting protrusions connected with outside of the chip groove or formed beyond the chip groove, and located outside of the two second side edges respectively, wherein the at least two limiting protrusions all extend toward the chip groove, and projections of the at least two limiting protrusions on a flat surface of the groove bottom of the chip groove is partially coincident with the groove bottom of the chip groove.

In some embodiments, the distance between a two first side edges is 54˜66 mm, a distance between the two second side edges is 36˜44 mm, and a height of the two second side edges relative to the groove bottom is 5.4˜6.6 mm.

In some embodiments, the limiting protrusion is a cylinder, and a bottom of the cylinder has a lateral notch facing towards the chip groove.

In some embodiments, the mounting portion includes a plurality of chip grooves formed on the inclined outer surface at equal intervals along a circumferential direction of the support portion.

In some embodiments, the support portion includes a first frustum cone, a cross-sectional diameter of a top end of the first frustum cone is smaller than that of a bottom of the first frustum cone, and the inclined outer surface is a side surface of the first frustum cone.

In some embodiments, fillets are provided between a side surface of the first frustum cone and each of a top end and a bottom of the first frustum cone respectively.

In some embodiments, the bottom of the first frustum cone has a inwardly concave cavity, such that the connecting portion is connected with or formed on an inner wall of the cavity, and the connecting portion includes an interface exposed relative to the bottom of the first frustum cone and configured to connect the rotor of the centrifuge.

In some embodiments, the connecting portion includes a second frustum cone having a hollow chamber and the interface, a top end of the second frustum cone is connected with or formed on the inner wall of the cavity, the interface is connected with or formed at a bottom of the second frustum cone and communicated with the hollow chamber of the second frustum cone, and a cross-sectional diameter of the top end of the second frustum cone is larger than that of the bottom of the second frustum cone.

In some embodiments, an inside diameter of the interface is 20˜30 mm, a difference between an outside diameter of the interface and the inside diameter of the interface is 2˜5 mm, and an exposed height of the interface relative to the bottom of the first frustum cone is 3˜7 mm.

In some embodiments, the microfluidic chip is a microarray digital polymerase chain reaction chip.

In one aspect of the present disclosure, a centrifuge is provided. The centrifuge includes: the centrifugal structure member of a microfluidic chip described above.

Therefore, according to the embodiments of the present disclosure, the rotor of the centrifuge is connected to the connecting portion, and the support portion with the inclined outer surface is detachably mounted on the support portion through the mounting portion. When the centrifugal structure member of the microfluidic chip is driven by the rotor of the centrifuge to rotate, the air bubbles inside the chip may be released from the chip based on the difference in a centrifugal force between the air bubbles and the reaction solution within the microfluidic chip, which helps to enhance the sample injection efficiency of the microfluidic chip and improve the reaction efficiency of the microfluidic chip.

### BRIEF DESCRIPTION OF THE ACCOMPANYING DRAWINGS

The accompanying drawings which constitute part of this specification, illustrate the exemplary embodiments of the present disclosure, and together with this specification, serve to explain the principles of the present disclosure.

The present disclosure may be more explicitly understood from the following detailed description with reference to the accompanying drawings, in which:

FIG. 1 is a schematic three-dimensional structural view of one embodiment of a centrifugal structure member of a microfluidic chip according to the present disclosure in an upper side viewing perspective;

FIG. 2 is a schematic three-dimensional structural view of one embodiment of a centrifugal structure member of a microfluidic chip according to the present disclosure in a lower side viewing perspective;

FIG. 3 is a schematic structural view of a mounting portion of one embodiment of a microfluidic chip centrifugal structural member according to the present disclosure;

FIG. 4 is a schematic cross-sectional view of the microfluidic chip in FIG. 3;

FIG. 5 is a schematic structural view of the microfluidic chip in FIG. 3;

FIG. 6 is a schematic structural view of one embodiment of the centrifugal structural member of the microfluidic chip according to the present disclosure in a front view perspective;

FIG. 7 is a schematic structural view of FIG. 6 in a left viewing perspective; and

FIG. 8 is a schematic structural view of FIG. 6 in a top view perspective.

It should be understood that the dimensions of various parts shown in the accompanying drawings are not drawn according to actual proportional relations. In addition, the same or similar components are denoted by the same or similar reference signs.

### DETAILED DESCRIPTION

Various exemplary embodiments of the present disclosure will now be described in detail with reference to the accompanying drawings. The description of the exemplary embodiments is merely illustrative and is in no way intended as a limitation to the present disclosure, its application or use. The present disclosure may be implemented in many different forms, which are not limited to the embodiments described herein. These embodiments are provided to make the present disclosure thorough and complete, and fully convey the scope of the present disclosure to those skilled in the art. It should be noticed that: relative arrangement of components and steps, material composition, numerical expressions, and numerical values set forth in these embodiments, unless specifically stated otherwise, should be explained as merely illustrative, and not as a limitation.

The use of the terms “first”, “second” and similar words in the present disclosure do not denote any order, quantity or importance, but are merely used to distinguish between different parts. A word such as “include”, “comprise” or variants thereof means that the element before the word covers the element (s) listed after the word without excluding the possibility of also covering other elements. The terms “up”, “down”, “left”, “right”, or the like are used only to represent a relative positional relationship, and the relative positional relationship may be changed correspondingly if the absolute position of the described object changes.

In the present disclosure, when it is described that a particular device is located between the first device and the second device, there may be an intermediate device between the particular device and the first device or the second device, and alternatively, there may be no intermediate device. When it is described that a particular device is connected to other devices, the particular device may be directly connected to the other devices without an intermediate device, and alternatively, may not be directly connected to the other devices but with an intermediate device.

All the terms (including technical and scientific terms) used in the present disclosure have the same meanings as understood by those skilled in the art of the present disclosure unless otherwise defined. It should also be understood that terms as defined in general dictionaries, unless explicitly defined herein, should be interpreted as having meanings that are consistent with their meanings in the context of the relevant art, and not to be interpreted in an idealized or extremely formalized sense.

Techniques, methods, and apparatus known to those of ordinary skill in the relevant art may not be discussed in detail, but where appropriate, these techniques, methods, and apparatuses should be considered as part of this specification.

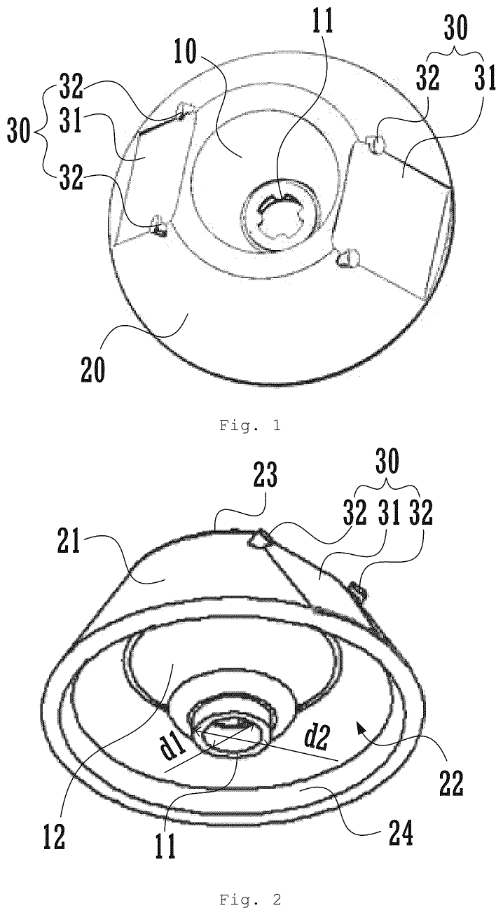

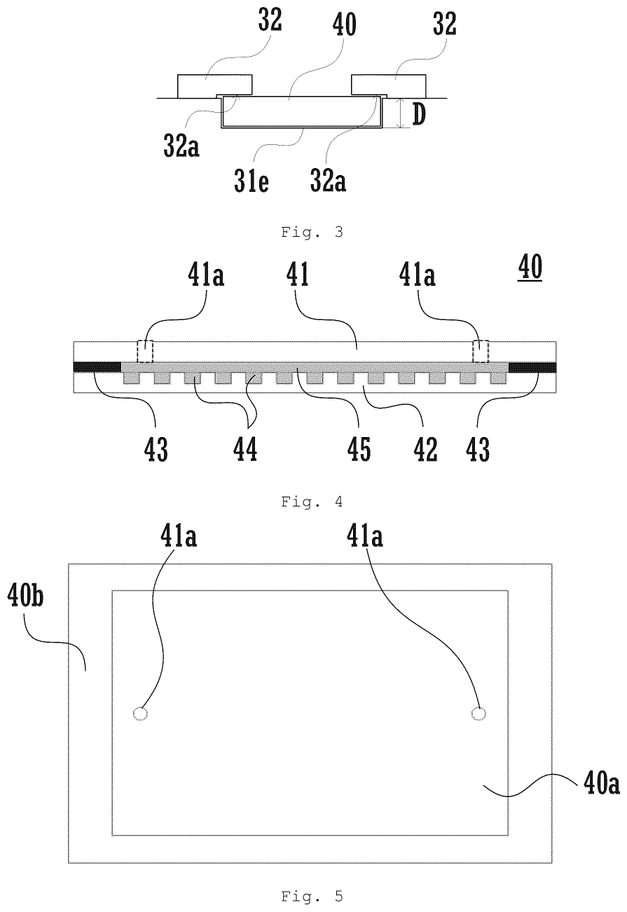

FIG. 1 is a schematic three-dimensional structural view of one embodiment of a centrifugal structure member of a microfluidic chip according to the present disclosure in an upper side viewing perspective. FIG. 2 is a schematic three-dimensional structural view of one embodiment of a centrifugal structure member of a microfluidic chip according to the present disclosure in a lower side viewing perspective. FIG. 3 is a schematic structural view of a mounting portion of one embodiment of a microfluidic chip centrifugal structural member according to the present disclosure.

Referring to FIGS. 1 and 2, in some embodiments, the centrifugal structure member of a microfluidic chip includes: a connecting portion 10, a support portion 20 and a mounting portion 30. The connecting portion 10 is configured to connect a rotor of a centrifuge. The centrifuge is a mechanical device that separate media with different specific gravities by a centrifugal force, which includes a rotor rotating according to a certain speed.

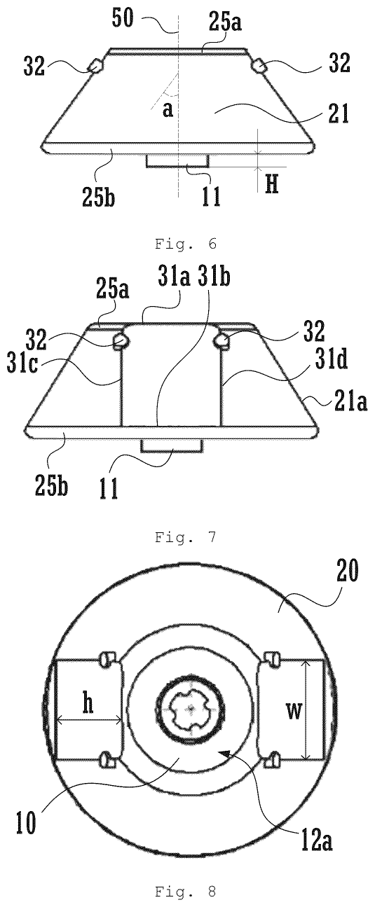

The support portion 20 is fixedly connected or integrally formed with the connecting portion 10, and has an inclined outer surface forming an included angle a (referring to FIG. 6) with a rotation axis 50 of the rotor of the centrifuge. The rotor may drive the support portion 20 to rotate about the rotation axis by driving the connecting portion 10 to rotate. The inclined outer surface here may be an arc-shaped surface or a flat surface. For example, the inclined outer surface may be an arc-shaped surface of a frustum cone, and the generatrix of the arc-shaped surface forms an included angle a with the rotation axis 50. The inclined outer surface may be at least one side surface of a truncated pyramid, and the side surface forms an included angle a with the rotation axis 50. The included angle a here is an acute angle, for example 15°˜75°.

The mounting portion 30 is connected or formed on the inclined outer surface, and configured to detachably mount the microfluidic chip 40 on the support portion 20. The microfluidic chip 40 may be mounted on the support portion 20 through the mounting portion 30, and may also be removed from the mounting portion 30. When the microfluidic chip 40 is mounted on the support portion 20 through the mounting portion 30, the microfluidic chip 40 may be also in such a state as to be inclined relative to the rotation axis 50.

In this embodiment, the rotor of the centrifuge is connected to the connecting portion, and the support portion with the inclined outer surface is detachably mounted on the support portion through the mounting portion. When the centrifugal structure member of the microfluidic chip is driven by the rotor of the centrifuge to rotate, the air bubbles inside the chip may be released from the chip based on the difference in a centrifugal force between the air bubbles and the reaction solution within the microfluidic chip, which helps to enhance the sample injection efficiency of the microfluidic chip and improve the reaction efficiency of the microfluidic chip.

Compared with the PCR tube centrifuge device in the related art, the microfluidic chip is mounted by the mounting portion connected with or formed on the inclined outer surface in this embodiment, which is not only more suitable for the shape of the microfluidic chip, but also convenient for observation. Moreover, such structure design is more simplified, and easily realized at low cost.

In some embodiments, the connecting portion 10, the support portion 20 and the mounting portion 30 may be manufactured independently and connected. In other embodiments, the connecting portion 10, the support portion 20 and the mounting portion 30 may be integrally formed. The materials of the connecting portion 10, the support portion 20 and the mounting portion 30 may be a rigid plastic such as polycarbonate or polyvinyl chloride.

Referring to FIGS. 1 and 2, in some embodiments, the mounting portion 30 includes: a chip groove 31. The chip groove 31 is formed on the inclined outer surface and configured to be embedded into by the microfluidic chip 40. In this way, when the microfluidic chip 40 is embedded into the chip groove 31, it may also be in an inclined state along with the inclination of the inclined outer surface. When the chip groove 31 rotates along with the support portion 20, the air bubbles in the microfluidic chip 40 can be released outward.

In order to stably fix the microfluidic chip 40 in the chip groove 31 during the centrifugation process, referring to FIGS. 1-3, in some embodiments, the mounting portion 30 further includes: a limiting protrusion 32. The limiting protrusion 32 is connected with outside of the chip groove or formed beyond the chip groove 31, and extend toward the chip groove 31. The projection of the limiting protrusion 32 on a flat surface of groove bottom of the chip groove 31 is partially coincident with the groove bottom 31e of the chip groove 31. The limiting protrusion 32 may constrain the microfluidic chip 40 in a direction perpendicular to the groove bottom, so as to avoid that the microfluidic chip 40 is thrown out of the chip groove 31 during the centrifugation process.

Referring to FIG. 1, in some embodiments, the mounting portion 30 includes a plurality of chip grooves 31 formed on the inclined outer surface at equal intervals along a circumferential direction of the support portion 20. For example, in FIG. 1, two chip grooves 31 are provided at 180º intervals along a circumferential direction of the support portion 20, which may realize simultaneous centrifuging operations of two microfluidic chips 40. In other embodiments, one chip groove 31, or three or more chip grooves 31 may be provided along a circumferential direction of the support portion 20. The number of chip grooves 31 may be determined according to the size of the chip and the size of the support portion 20. In addition, a plurality of chip grooves 31 are provided at equal intervals so that the centrifugation process may be more balanced.

Referring to FIGS. 1 and 3, in some embodiments, the limiting protrusion 32 is a cylinder including a lateral notch 32a facing towards the chip groove 31 at the bottom. When the microfluidic chip 40 is embedded into the chip groove 31, the microfluidic chip 40 is constrained in a direction perpendicular to the groove bottom 31e by the groove bottom 31e of the chip groove 31 and the lateral notch 32a. During manufacturing, it is possible to integrally form the cylindrical limiting protrusion 32 and the support portion 20 firstly, then form the chip groove 31 by machining the inclined outer surface of the support portion 20, and at the same time form a lateral notch 32a on the bottom side of the limiting protrusion 32.

FIG. 4 is a schematic cross-sectional view of the microfluidic chip in FIG. 3. FIG. 5 is a schematic structural view of the microfluidic chip in FIG. 3.

In some embodiments, the microfluidic chip 40 is a microarray digital polymerase chain reaction (PCR) chip. Referring to FIGS. 4 and 5, the microarray digital PCR chip may include two layers of glass substrates 41 and 42. The two layers of glass substrates 41 and 42 are adhered each other and sealed by an encapsulating adhesive layer 43, and form a thin cavity 45 sandwiched between the two layers of glass substrates 41 and 42.

In FIG. 5, the area 40b corresponding to the encapsulating adhesive layer of the microarray digital PCR chip is located in a periphery of the reaction chamber area 40a.

In FIG. 4, the glass substrate 41 includes two sample injection holes 41a, and the glass substrate 42 has a plurality of hollow chambers 44 arranged in an array at positions corresponding to the reaction chamber area 40a. The plurality of hollow chambers 44 are located on one side of the thin cavity 45, and communicate with the thin cavity 45. When the microarray digital PCR chip performs sample injection, the PCR reaction solution and the oil phase enter the inside of the chip through one sample injection hole 41a sequentially, and are filled into the thin cavity 45 and the plurality of hollow chambers 44, and excessive liquid may flow out from another sample injection hole 41a. In the state shown in FIG. 4, the PCR reaction solution is located in the plurality of hollow chambers 44. The oil phase is located in the thin cavity 45 for enclosing the PCR reaction solution in the plurality of hollow chambers 44. The oil phase may include mineral oil, electronic fluoride, and the like.

When the microarray digital PCR chip performs sample injection, first of all, the chip chamber is filled with the reaction solution and the oil phase as much as possible by a conventional sample injection method, and a small amount of air bubbles might be distributed throughout the array chamber; and subsequently one sample injection hole is enclosed using a material such as UV curing adhesive. Then, the microarray digital PCR chip is mounted into the chip groove 31, so that the unenclosed sample injection hole 41a of the microarray digital PCR chip is more proximate to the rotation axis 50, and the enclosed sample injection hole 41a is more away from the rotation axis 50.

In this way, when the inclined microarray digital PCR chip rotates along with the support portion 20, the PCR reaction solution and the oil phase with a mass greater than that of the air bubbles obtain a higher centrifugal force and thus move to one end of the microarray digital PCR chip more away from the rotation axis 50, while the air bubbles move to one end of the microarray digital PCR chip that is more adjacent to the rotation axis 50, and released from the sample injection hole 41a at this end. This helps to enhance the sample injection efficiency of the chip, and improve the reaction efficiency of the chip, thereby improving the amplification efficiency and detection accuracy of the chip, and further enhancing the application value of the microarray digital PCR chip in the field of molecular diagnosis.

FIG. 6 is a schematic structural view of one embodiment of the centrifugal structural member of the microfluidic chip according to the present disclosure in a front view perspective. FIG. 7 is a schematic structural view of FIG. 6 in a left viewing perspective. FIG. 8 is a schematic structural view of FIG. 6 in a top view perspective.

Referring to FIGS. 6 and 7, in some embodiments, the support portion 20 includes a first frustum cone 21, wherein the cross-sectional diameter of the top end 23 of the first frustum cone 21 is smaller than that of the bottom 24 of the first frustum cone 21, and the inclined outer surface is a side surface 21a of the first frustum cone 21. When the centrifugal structure member of the microfluidic chip performs a centrifuging operation, the axis of the first frustum cone 21 is coincident with the rotation axis 50 of the rotor, and the top end 23 of the first frustum cone 21 is located on the upper side of the bottom 24 of the first frustum cone 21. In this way, it is possible that the formed chip groove 31 is in such a state as to be inclined along an upward direction.

In FIGS. 6 and 7, a fillet 25a (for example, a radius of 2.5 mm, or the like) is provided between the top end 23 of the first frustum cone 21 and the side surface of the first frustum cone 21, and a fillet 25b is provided between the bottom 24 of the first frustum cone 21 and the side surface of the first frustum cone 21, which may make the upper and lower edges of the first frustum cone 21 more smooth so as to facilitate the operation, and it is not easily to do harm to the chip or the operator.

Referring to FIG. 2, in some embodiments, the bottom of the first frustum cone 21 has an inwardly concave cavity 22, the connecting portion 10 is connected with or formed on the inner wall of the cavity 22, and the connecting portion 10 includes an interface 11 exposed relative to the bottom of the first frustum cone 21 and configured to connect the rotor of the centrifuge. The connecting portion is connected with or formed on the inner wall of the cavity 22, so that it is possible to make a more compact structure of the connecting portion and the support portion, and reduce the occupied space. Moreover, the cavity may also make the overall weight lighter and reduce the material consumption. The interface 11 is exposed relative to the bottom of the first frustum cone 21, so that the structure such as the support portion is lifted to avoid interference with other members of the centrifuge during the centrifuging operation process.

For example, the diameter of the top end of the first frustum cone 21 is optionally 53.67 mm, and the diameter of the bottom is optionally 115.61 mm; the height of the first frustum cone 21 along a direction parallel to the rotation axis 50 is optionally 43.82 mm; and the first frustum cone 21 has a cavity, wherein the thicknesses of the respective cavity walls are optionally about 4 mm.

Referring to FIGS. 1 and 2, in some embodiments, the connecting portion 10 includes a second frustum cone 12 having a hollow chamber 12a and the interface 11, wherein the top end of the second frustum cone 12 is connected with or formed on the inner wall of the cavity 22. The interface 11 is connected with or formed at the bottom of the second frustum cone 12 and communicates with the hollow chamber 12a of the second frustum cone 12. The cross-sectional diameter of the top end of the second frustum cone 12 is greater than that of the bottom of the second frustum cone 12. In this way, it is possible to realize such a structure that the first frustum cone and the second frustum cone with the cross-sectional diameters varying in opposite directions are snap-fit to each other. Such structure enables the interface 11 with a small size to be matched with the rotor of the centrifuge, and it is also possible to obtain a large cavity proportion, thereby further reducing the overall weight.

Referring to FIGS. 2 and 6, in some embodiments, the inside diameter r1 of the interface 11 is 20˜30 mm, for example 24 mm or the like, and the difference between the outside diameter r2 of the interface 11 and the inside diameter r1 of the interface 11 is 2˜5 mm, for example, the outside diameter r2 is 26.72 mm, or the like. The exposed height H of the interface 11 relative to the bottom of the first frustum cone 21 is 3˜7 mm, for example, 5 mm or the like.

Referring to FIGS. 1, 7 and 8, in some embodiments, the chip groove 31 is rectangular, and has two first side edges 31a and 31b that are perpendicular to the rotation axis 50 and opposite to each other, and two second side edges 31c and 31d that are perpendicular to the first side edges 31a and 31b and opposite to each other. The height of a first one of the two first side edges 31a and 31b relative to the groove bottom 31e of the chip groove 31 is lower than that of a second one relative to the groove bottom 31e. In this way, when the microfluidic chip 40 is mounted or removed, the microfluidic chip 40 may enter into or exit from the chip groove 31 from the first side edge 31a, while the first side edge 31b restricts the microfluidic chip 40 so that it cannot escape from the chip groove 31 from this side.

In order to make it easier for the microfluidic chip 40 to enter into or exit from the chip groove 31, referring to FIG. 7, in some embodiments, the first one of the two first side edges 31a and 31b is flush with the top end of the support portion 20. In addition, the maximum distance between the first side edge 31b and the outer edge of the bottom surface of the first frustum cone 21 corresponding to the first side edge 31b is 3.26 mm.

In FIGS. 1 and 8, the chip groove 31 is formed by being recessed inwardly on the side surface 21a of the first frustum cone 21, so that the first side edge 31a is more proximate to the rotation axis 50 than the first side edge 31b. In this way, when the support portion 20 rotates, the lower end of the microfluidic chip 40 withstands the first side edge 31b tightly, while the air bubbles move to one side proximate to the first side edge 31a, and are released outwards from the sample injection hole 41a on this side.

Referring to FIGS. 1 and 7, in some embodiments, the mounting portion 30 further includes: at least two limiting protrusions 32. The at least two limiting protrusions 32 are connected with outside of the chip groove 31 or formed beyond the chip groove 31, and respectively located outside the two second side edges 31c and 31d. In other words, some of the at least two limiting protrusions 32 is located outside the second side edge 31c, and the other are located outside the second side edge 31d. For example, in FIG. 7, one limiting protrusion 32 is located on the left side of the second side surface 31c, and another limiting protrusion 32 is located on the right side of the second side edge 31d.

The at least two limiting protrusions 32 all extend toward the chip groove 31, and their projections on a flat surface of groove bottom of the chip groove 31 is partially coincident with the groove bottom 31e of the chip groove 31. In this way, the microfluidic chip 40 is restricted by the at least two limiting protrusions 32, so that the microfluidic chip 40 is more stable during the centrifuging process, and is prevented from being thrown out during the centrifuging. In other embodiments, the mounting portion 30 may include one limiting protrusion, or the at least two limiting protrusions 32 are all outside one second side edges 31c or 31d.

The size of the chip groove 31 may be designed according to the size of the microfluidic chip. Referring to FIGS. 3 and 8, for example, the distance h between the two first side edges 31a and 31b is 54˜66 mm, which is optionally 60 mm; the distance w between the two side edges 31c and 31d is 36˜44 mm, which is optionally 40 mm; the height of the two second side edges 31c and 31d relative to the groove bottom 31e is 5.4˜6.6 mm, which is optionally 6 mm. The diameter of the bottom surface of the cylinder of the limiting protrusion 32 is optionally 6 mm.

The embodiments of the above-described centrifugal structure member of a microfluidic chip of the present disclosure may be applied to a plurality of centrifugal devices, for example a small centrifuge, which releases the air bubbles from the chip chamber by way of the principles of different centrifugal forces on the liquid and the air bubbles. Therefore, the present disclosure also provides a centrifuge, which includes any one of the foregoing embodiments of the centrifugal structure member of a microfluidic chip. In the centrifuge, a rotor and a driving mechanism for driving the rotor to rotate may also be included.

Hereto, various embodiments of the present disclosure have been described in detail. Some details well known in the art are not described in order to avoid obscuring the concept of the present disclosure. According to the above description, those skilled in the art would fully understand how to implement the technical solutions disclosed here.

Although some specific embodiments of the present disclosure have been described in detail by way of examples, those skilled in the art should understand that the above examples are only for the purpose of illustration but not for limiting the scope of the present disclosure. It should be understood by those skilled in the art that modifications to the above embodiments and equivalently substitution of part of the technical features may be made without departing from the scope and spirit of the present disclosure. The scope of the present disclosure is defined by the appended claims.

1. A centrifugal structure member of a microfluidic chip, comprising:
a connecting portion configured to connect a rotor of a centrifuge;
a support portion fixedly connected or integrally formed with the connecting portion, and having an inclined outer surface forming an included angle with a rotation axis of the rotor of the centrifuge; and
a mounting portion connected with or formed on the inclined outer surface, and configured to detachably mount the microfluidic chip on the support portion.

2. The centrifugal structure member of a microfluidic chip according to claim 1, wherein the mounting portion comprises:
a chip groove formed on the inclined outer surface, and configured to be embedded into by the microfluidic chip.

3. The centrifugal structure member of a microfluidic chip according to claim 2, wherein the mounting portion further comprises:
a limiting protrusion connected with outside of the chip groove or formed beyond the chip groove, and extending toward the chip groove,
wherein a projection of the limiting protrusion on a flat surface of groove bottom of the chip groove is partially coincident with the groove bottom of the chip groove.

4. The centrifugal structure member of a microfluidic chip according to claim 2, wherein the chip groove is rectangular and has two first side edges perpendicular to the rotation axis and opposite to each other, and two second side edges perpendicular to the two first side edges and opposite to each other, and a height of a first one of the two first side edges relative to the groove bottom of the chip groove is lower than that of a second one of the two first side edges relative to the groove bottom.

5. The centrifugal structure member of a microfluidic chip according to claim 4, wherein the first one of the two first side edges is flush with a top end of the support portion.

6. The centrifugal structure member of a microfluidic chip according to claim 4, wherein the mounting portion further comprises:
at least two limiting protrusions connected with outside of the chip groove or formed beyond the chip groove, and located outside of the two second side edges respectively,
wherein the at least two limiting protrusions all extend toward the chip groove, and projections of the at least two limiting protrusions on a flat surface of the groove bottom of the chip groove is partially coincident with the groove bottom of the chip groove.

7. The centrifugal structure member of a microfluidic chip according to claim, wherein a distance between the two first side edges is 54˜66 mm, a distance between the two second side edges is 36˜44 mm, and a height of the two second side edges relative to the groove bottom is 5.4˜6.6 mm.

8. The centrifugal structure member of a microfluidic chip according to claim 3, wherein the limiting protrusion is a cylinder, and a bottom of the cylinder has a lateral notch facing towards the chip groove.

9. The centrifugal structure member of a microfluidic chip according to claim 2, wherein the mounting portion comprises a plurality of chip grooves formed on the inclined outer surface at equal intervals along a circumferential direction of the support portion.

10. The centrifugal structure member for a microfluidic chip according to claim 1, wherein the support portion comprises a first frustum cone, a cross-sectional diameter of a top end of the first frustum cone is smaller than that of a bottom of the first frustum cone, and the inclined outer surface is a side surface of the first frustum cone.

11. The centrifugal structure member of a microfluidic chip according to claim 10, wherein fillets are provided between a side surface of the first frustum cone and each of a top end and a bottom of the first frustum cone respectively.

12. The centrifugal structure member of a microfluidic chip according to claim 10, wherein the bottom of the first frustum cone has an inwardly concave cavity, the connecting portion is connected with or formed on an inner wall of the cavity, and the connecting portion comprises an interface exposed relative to the bottom of the first frustum cone and configured to connect the rotor of the centrifuge.

13. The centrifugal structure member of a microfluidic chip according to claim 12, wherein the connecting portion comprises a second frustum cone having a hollow chamber and the interface, a top end of the second frustum cone is connected with or formed on the inner wall of the cavity, the interface is connected with or formed at a bottom of the second frustum cone and communicated with the hollow chamber of the second frustum cone, and a cross-sectional diameter of the top end of the second frustum cone is larger than that of the bottom of the second frustum cone.

14. The centrifugal structure member of a microfluidic chip according to claim 12, wherein an inside diameter of the interface is 20˜30 mm, a difference between an outside diameter of the interface and the inside diameter of the interface is 2˜5 mm, and an exposed height of the interface relative to the bottom of the first frustum cone is 3˜7 mm.

15. The centrifugal structure member of a microfluidic chip according to claim 1, wherein the microfluidic chip is a microarray digital polymerase chain reaction chip.

16. A centrifuge comprising:
the centrifugal structure member of a microfluidic chip according to claim 1.
